(19) United States
(12) Patent Application Publication
(10) Pub. No.: US 2026/0177568 A1
(43) Pub. Date: Jun. 25, 2026

(54) TESTS AND METHODS FOR RAPID AND QUANTITATIVE DETECTION OF AN ACETYLCHOLINE RECEPTOR LIGAND

(71) Applicants: Lance Philip Ford, Austin, TX (US); Joseph Francis Krebs, Austin, TX (US)

(72) Inventors: Lance Philip Ford, Austin, TX (US); Joseph Francis Krebs, Austin, TX (US)

(21) Appl. No.: 19/401,318

(22) Filed: Nov. 25, 2025

Related U.S. Application Data
(60) Provisional application No. 63/729,119, filed on Dec. 6, 2024.

(51) Int. Cl. G01N 33/94; G01N 33/543; G01N 33/566

(52) U.S. Cl. CPC G01N 33/944; G01N 33/54388; G01N 33/566; G01N 2333/70571

(57) ABSTRACT

The present invention provides lateral flow assay devices and methods for detecting nicotinic acetylcholine receptor ligands in liquid samples. The device comprises a porous strip with multiple zones, including a sample deposition zone, a test visualization zone containing an immobilized nicotine conjugate, a control zone, and an absorbent zone. A recombinant acetylcholine receptor protein serves as a mobile agent, either pre-deposited on the strip or added to the sample. The recombinant protein comprises a ligand-binding domain of a nicotinic acetylcholine receptor conjugated to a visually-detectable reporter forming a receptor-reporter conjugate. When nicotinic acetylcholine receptor ligands are present in the sample, they competitively bind to the recombinant receptor protein, reducing binding to the immobilized nicotine conjugate and producing a visually detectable signal inversely proportional to ligand concentration. The invention provides rapid, simple, and field-deployable detection of receptor ligands in environmental samples, including cyanobacterial neurotoxins in surface waters, and biological samples for pharmacological and toxicological applications.

### CROSS-REFERENCE DATA

This patent application claims a priority date benefit from a co-pending U.S. Provisional Patent Application No. 63/729,119 entitled “Rapid and quantitative methods for detection of Dihydroanatoxin-a or other compounds associated with recombinant acetylcholine receptor using recombinant protein and nicotine conjugate” and filed on Dec. 6, 2024, which is incorporated herein by reference in its entirety.

### SEQUENCE LISTING XML

The contents of the electronic sequence listing (Acetylcholine_Binding_Receptor.xml; Size: 2,259 bytes; and Date of Creation: Nov. 25, 2025) is herein incorporated by reference in its entirety.

### BACKGROUND

Without limiting the scope of the invention, its background is described in connection with diagnostic and analytical assays for detecting, identifying, or quantifying compounds that bind to nicotinic acetylcholine receptors (nAChRs). More particularly, the invention concerns devices and methods for rapidly detecting nicotinic acetylcholine receptor ligands in environmental or biologically-derived liquid samples. The invention provides lateral flow assay devices comprising recombinant acetylcholine receptor proteins as mobile agents and nicotine conjugates as immobilized agents, as well as related test devices and methods employing these components for competitive binding assays. The invention finds utility in fields including biomedical research, pharmacology, toxicology, neurobiology, environmental testing, and water quality monitoring.

Acetylcholine Receptors and their Biological Importance

Acetylcholine receptors (AChRs) are integral membrane proteins that mediate the physiological actions of the neurotransmitter acetylcholine. Two principal classes of receptors have been identified: nicotinic acetylcholine receptors (nAChRs), which are ligand-gated ion channels, and muscarinic acetylcholine receptors (mAChRs), which are G-protein-coupled receptors. Nicotinic acetylcholine receptors are widely distributed in the neuromuscular junction, autonomic ganglia, and central nervous system, where they play essential roles in neurotransmission, muscle contraction, cognitive function, and autonomic regulation.

Because of their central role in signaling pathways, acetylcholine receptors are critical targets in medicine and pharmacology. Alterations in receptor function or expression are implicated in disorders such as Alzheimer's disease, myasthenia gravis, schizophrenia, nicotine dependence, and neurodegenerative conditions. In addition, many neurotoxins and environmental pesticides target nicotinic acetylcholine receptors as nicotinic acetylcholine receptor ligands. For example, cyanobacteria that commonly grow in surface waters, including Anabaena flos-aquae and Microcoleus anatoxicus, produce the neurotoxins anatoxin-a and dihydroanatoxin-a, which are potent agonists of nicotinic acetylcholine receptors. It would be highly useful to have rapid tests for these cyanobacterial neurotoxins in recreational and other surface waters. Accordingly, methods and devices for detecting or quantifying nicotinic acetylcholine receptor ligands that bind to these receptors are of great importance in drug discovery, medical diagnostics, toxicology, and environmental safety testing.

### Existing Methods to Detect Acetylcholine Receptor Ligands

Numerous analytical approaches have been developed to study receptor-ligand interactions involving acetylcholine receptors. Traditional radioligand binding assays use isotopically labeled ligands such as [³H]-nicotine or [¹²⁵1]-α-bungarotoxin to quantify receptor binding and affinity. While radioligand assays provide high sensitivity and reliable kinetic data, they require radioactive materials, specialized facilities, and complex waste disposal, which limit their use to controlled laboratory settings and trained personnel.

To avoid radioactivity, fluorescent and luminescent binding assays have been developed, employing ligands or receptors conjugated with optical reporter molecules. These methods are non-radioactive and suitable for microplate, imaging, or flow cytometry formats. However, these methods often require sophisticated optical detection instrumentation and are often prone to photobleaching or signal quenching. Also, the required conjugation of bulky signaling moieties to the ligands may adversely affect their binding properties to the receptors.

Electrophysiological techniques, such as patch-clamp and voltage-clamp assays, permit direct measurement of ion channel activity mediated by nicotinic acetylcholine receptors. These approaches provide functional information with high temporal resolution, but are labor-intensive, technically demanding, low-throughput, and generally unsuitable for routine screening or field applications.

Label-free biosensor systems, including surface plasmon resonance (SPR), biolayer interferometry (BLI), and related optical techniques, can monitor ligand-receptor interactions in real time without chemical labels. While these systems yield detailed kinetic data, they require expensive instrumentation, complex surface immobilization procedures, and highly purified receptor preparations, limiting their practical use for distributed testing.

Cell-based assays employing receptor-expressing lines offer physiologically relevant contexts for ligand interaction studies. However, these methods exhibit biological variability, require careful maintenance of live cell cultures, are time-consuming, and are difficult to standardize across laboratories. The sensitivity of living systems to environmental factors further complicates reproducibility and quantitative analysis. In addition, in vivo mouse bioassays have historically been used to assess the biological activity or toxicity of acetylcholine receptor ligands. While these assays reflect whole-organism pharmacodynamics, they are slow, costly, ethically constrained, and poorly suited for high-throughput or quantitative screening, with species-specific differences limiting relevance to human pharmacology.

Modern instrument-based analytical techniques, including liquid chromatography coupled with tandem mass spectrometry (LC-MS/MS), have also been utilized to detect and quantify acetylcholine receptor ligands or related compounds. While LC-MS/MS offers high sensitivity and specificity, it generally detects total compound rather than functional receptor binding activity. These methods also require expensive instruments, highly trained operators, complex sample preparation, and are not compatible with rapid, field-deployable, or point-of-care formats.

Challenges with Isolated Receptor-Based Assays

To overcome some of these limitations, isolated receptors or receptor fragments have been explored as reagents for rapid competitive binding assays, such as microplate format assays. In such assays, a receptor or receptor fragment—either immobilized or conjugated to a reporter group—can compete with sample ligands for binding, allowing for quantitative detection of receptor-binding compounds without the need for live cells or radioactive tracers. However, producing active mammalian receptor reagents poses significant challenges. Mammalian nicotinic acetylcholine receptors are multimeric, membrane-embedded proteins, and maintaining their native conformation and ligand-binding activity after extraction from biological membranes or recombinant expression is technically difficult.

Recombinant expression systems often yield misfolded, aggregated, or inactive receptor proteins, particularly for full-length transmembrane subunits, and purification protocols can be inefficient, labor-intensive, and costly. Even when active fragments are obtained, they may require detergents or membrane-like environments to maintain activity, and they often exhibit decreased activity and stability, reducing reproducibility and shelf-life and limiting their utility as reagents in practical, rapid assay formats.

Nicotinic acetylcholine receptors are heteropentameric ligand-gated ion channels composed of multiple subunits. Their folding, assembly, and surface trafficking are complex and tightly regulated processes that often require specific cellular environments or tissue-specific factors for functional expression. Many subtypes fail to create functional receptor channels when expressed in standard recombinant systems, impeding reliable in vitro use. Achieving stable and high-yield expression of recombinant functional mammalian nicotinic acetylcholine receptors in non-native cell lines (such as Chinese hamster ovary (CHO) cells) is a major hurdle. The receptors are prone to misfolding and degradation by cellular quality control mechanisms (e.g., endoplasmic reticulum-associated degradation (ERAD)). Soluble peripheral acetylcholine binding proteins, such as the AChBP protein from the invertebrate Aplysia californica, may be potential reagents because they are soluble homopentamers that bind some nicotinic ligands, but their ability to bind important toxins, such as dihydroanatoxin, and their adaptability to serve in lateral flow assays is unknown

### Need for Improved Detection Methods

Because of these limitations, there remains a need for simple, rapid, sensitive, and robust assays that can reliably detect nicotinic acetylcholine receptor ligands binding to acetylcholine receptors in liquid samples, without reliance on radioactive materials, live cells, or high-cost instrumentation. Ideally, such assays would be portable, scalable, and adaptable to a variety of diagnostic and analytical formats, including lateral flow assay devices employing porous strips, test strips, and microwell devices, while employing stable and reproducible recombinant acetylcholine receptor proteins as mobile agents or receptor-based reagents. Furthermore, such devices and methods should provide visually detectable signals that allow for rapid qualitative or semi-quantitative assessment of nicotinic acetylcholine receptor ligand concentration in liquid samples, with the concentration of the nicotinic acetylcholine receptor ligand being inversely proportional to the intensity or strength of the visually detectable signal generated by receptor-reporter conjugates binding to immobilized agents such as nicotine conjugates.

### SUMMARY

Accordingly, it is an object of the present invention to overcome these and other drawbacks of the prior art by providing novel tests and methods capable of identifying, detecting and quantifying compounds that bind to acetylcholine receptors in a manner that is rapid and compatible with contemporary diagnostic applications.

The present invention addresses this need by providing a novel competitive binding assay in which a recombinant acetylcholine receptor protein conjugated to a reporter molecule (i.e., a receptor-reporter conjugate) interacts with an immobilized receptor ligand. The invention enables detection of nicotinic acetylcholine receptor ligands in liquid samples through modulation of a visually detectable signal, providing a versatile platform suitable for both lateral flow assay devices and microwell formats, and overcoming many of the disadvantages associated with existing acetylcholine receptor ligand detection methods.

In one aspect, the invention provides a lateral flow assay device for detecting a presence in a liquid sample of a nicotinic acetylcholine receptor ligand. The lateral flow assay device comprises a porous strip with a plurality of zones located along thereof, including: a first zone for depositing the liquid sample; a third zone comprising, in turn, a test visualization zone comprising a nicotine conjugate as an immobilized agent, and a control zone; and a fourth zone configured to absorb the liquid sample therein. The lateral flow assay device further comprises a recombinant acetylcholine receptor protein as a mobile agent.

In certain embodiments, the recombinant acetylcholine receptor protein is present on a second zone of the porous strip between the first zone and the third zone thereof. Alternatively, the recombinant acetylcholine receptor protein may be configured to be present in or added to the liquid sample prior to depositing the liquid sample onto the first zone.

The acetylcholine binding protein may be conjugated to a detectable reporter (such as a visually- or otherwise detectable reporter) to form a receptor-reporter conjugate. In embodiments, the reporter comprises a colored particle, a fluorescent marker, a magnetic particle, or a combination thereof. In particular embodiments, the reporter comprises a colored particle selected from the group consisting of gold nanoparticles, latex beads, carbon particles, selenium particles, and colored liposomes.

The nicotine conjugate comprises nicotine or a nicotine analog or a nicotine metabolite conjugated to a carrier molecule. The term “nicotine conjugate” is used broadly herein to also include other nicotinic acetylcholine agonists or nicotinic acetylcholine antagonists, or derivatives thereof.

In embodiments, the carrier molecule comprises a protein, a polymer, or a surface-bound molecule. In particular embodiments, the nicotine conjugate comprises nicotine or a nicotine analog conjugated to bovine serum albumin (BSA), keyhole limpet hemocyanin (KLH), ovalbumin, or a synthetic polymer.

The control zone of the third zone may comprise a capture agent capable of binding the receptor-reporter conjugate independently of nicotinic acetylcholine receptor ligand binding. Other control line designs are also conceived by the present invention, as described below.

In use, when the liquid sample is deposited onto the first zone and flows through the porous strip, if a nicotinic acetylcholine receptor ligand is present in the liquid sample, it binds to the recombinant acetylcholine receptor protein (the mobile agent), thereby reducing binding of the recombinant acetylcholine receptor protein to the nicotine conjugate (the immobilized agent) at the test visualization zone. Consequently, the visually detectable signal at the test visualization zone is inversely proportional to the concentration of nicotinic acetylcholine receptor ligand in the liquid sample: higher concentrations of nicotinic acetylcholine receptor ligand result in weaker signals at the test visualization zone, while lower concentrations result in stronger signals.

In another aspect, the invention provides a test device for detecting the presence of a nicotinic acetylcholine receptor ligand in a liquid sample. The test device comprises a solid support, a nicotine conjugate immobilized on the solid support, and a receptor-reporter conjugate comprising a recombinant acetylcholine receptor protein conjugated to a visually-detectable reporter. The recombinant acetylcholine receptor protein comprises a ligand-binding domain of a nicotinic acetylcholine receptor and is capable of binding to the nicotine conjugate on the solid support to produce a visually detectable signal. When the nicotinic acetylcholine receptor ligand is present in the liquid sample, it binds to the recombinant acetylcholine receptor protein, thereby reducing binding of the receptor-reporter conjugate to the nicotine conjugate and correspondingly reducing the visually detectable signal, such that the concentration of the nicotinic acetylcholine receptor ligand is inversely proportional to the intensity or strength of the visually detectable signal.

In embodiments, the solid support comprises a membrane, a lateral flow strip, a microwell plate, a dipstick, a bead, a microarray surface, or a combination thereof. The test device may be configured as a lateral flow assay device or as a microwell-based assay device.

In a further aspect, the invention provides methods for detecting a presence of a nicotinic acetylcholine receptor ligand in a liquid sample using the lateral flow assay devices and test devices described herein. The methods generally comprise contacting the liquid sample with a recombinant acetylcholine receptor protein (or receptor-reporter conjugate), allowing competitive binding to occur between any nicotinic acetylcholine receptor ligand present in the sample and an immobilized nicotine conjugate, and detecting a visually detectable signal that is inversely proportional to the concentration of nicotinic acetylcholine receptor ligand in the liquid sample.

The invention thus provides simple, rapid, cost-effective, and field-deployable devices and methods for detecting nicotinic acetylcholine receptor ligands in liquid samples, including environmental samples such as recreational surface waters potentially contaminated with cyanobacterial neurotoxins (e.g., anatoxin-a and dihydroanatoxin-a), as well as biological samples for pharmacological, toxicological, or diagnostic applications.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates the specific binding of recombinant acetylcholine receptor protein conjugated to reporter nanoparticles to an immobilized nicotine conjugate (nicotine-N-β-D glucuronide-protein conjugate) within the test visualization zone of a lateral flow assay device. The left strip demonstrates that, in the absence of a nicotinic acetylcholine receptor ligand (e.g., nicotine, 0 ppm), the receptor-reporter conjugates visibly accumulate in the test visualization zone, yielding a visually detectable signal. The right strip demonstrates that when nicotine is present in the running buffer (25 ppm), binding of the receptor-reporter conjugates to the immobilized agent is inhibited, resulting in reduced signal intensity at the test visualization zone.

FIG. 2 shows the use of the lateral flow assay device within a cassette housing for detecting anatoxin-a in water samples. The right device, exposed to a water sample containing 100 ppb anatoxin-a, shows a substantial decrease in visually detectable signal intensity at the test line compared to a control sample with no anatoxin-a (left device). The control line remains unaffected by the presence of anatoxin-a.

FIG. 3 depicts the detection of dihydroanatoxin using lateral flow assay devices. The test using a water sample containing 32 ppb dihydroanatoxin (right strip) results in a marked reduction of signal intensity in the test visualization zone relative to a negative control sample (left strip).

FIG. 4 illustrates the detection of nicotine in water samples using lateral flow assay devices. The right strip, tested with a sample containing 17 ppm nicotine, exhibits diminished signal intensity at the test visualization zone compared to the left strip with no nicotine present.

FIG. 5 demonstrates the detection of a neonicotinoid pesticide (imidacloprid) in aqueous samples using a lateral flow assay device. Exposure to a sample containing 1250 ppb imidacloprid (right strip) leads to a pronounced reduction in the visually detectable signal at the test line compared to a control sample (left strip).

FIG. 6 portrays the detection of nicotine in water using a microwell assay format (NG-BSA-coated 96-well plates) and a receptor-reporter conjugate linked to an enzyme (e.g., horseradish peroxidase). The binding of the recombinant acetylcholine receptor protein to immobilized nicotine conjugate protein is proportional to an absorbance signal recorded at 450 nm. Introducing 0.5 μg/μL nicotine into the sample (right) lowers the enzymatic signal compared to a control sample without nicotine (left), reflecting competitive inhibition of receptor binding.

### DETAILED DESCRIPTION OF PREFERRED EMBODIMENTS OF THE INVENTION

The following description sets forth various examples along with specific details to provide a thorough understanding of the claimed subject matter. It will be understood by those skilled in the art, however, that claimed subject matter may be practiced without one or more of the specific details disclosed herein. Further, in some circumstances, well-known methods, procedures, systems, components, and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

For the purposes of this disclosure, the term “receptor” refers to a protein that specifically binds ligands that share structural similarity, such as acetylcholine and its structurally related agonists or antagonists.

In various embodiments, the present invention may provide assay devices and methods configured for detecting, identifying, or quantifying the presence of a nicotinic acetylcholine receptor ligand in a liquid sample. The invention may employ a lateral flow assay device including a porous strip comprising a plurality of zones, such as a first zone configured for depositing the liquid sample, a second zone that may provide a storage area for a recombinant acetylcholine receptor protein as a mobile agent, a third zone comprising a test visualization zone that may include a nicotine conjugate as an immobilized agent, and a control zone, as well as a fourth zone for absorbing the liquid sample. The porous strip may be formed from nitrocellulose or other suitable porous material to promote capillary-driven flow of the liquid sample.

The recombinant acetylcholine receptor protein may function as the receptor component in a competitive binding assay. The protein may be produced by recombinant expression and purified from, for example, insect or bacterial cell culture. In certain embodiments, the recombinant acetylcholine receptor protein may comprise an acetylcholine-binding protein (AChBP), which may be expressed as a soluble homopentamer produced from a single polypeptide. The recombinant acetylcholine receptor protein serves as a mobile agent capable of binding to the nicotinic acetylcholine receptor ligand. The recombinant acetylcholine receptor protein may be provided in a liquid or lyophilized form and may be introduced to the liquid sample either prior to application or through a dedicated sample pad or zone on the porous strip. The liquid sample may comprise various matrices, such as water, buffer, environmental or biological fluids, and may contain the analyte(s) of interest.

### Production of Recombinant Acetylcholine Receptor Protein

The recombinant acetylcholine receptor protein may be produced using insect cell culture, such as High Five cells, using methods known in the art. This approach may offer several advantages over other expression systems. First, the active recombinant acetylcholine receptor protein may be a soluble homopentamer produced from a single polypeptide. Second, the protein may be expressed in an optimized cell culture system designed to produce correctly folded protein with properly formed disulfide bonds. A secretion signal sequence, for example the signal peptide derived from the gp67 envelope glycoprotein of Autographa californica nuclear polyhedrosis virus, may be fused to the N-terminus of the protein to promote efficient secretion and proper disulfide bond formation. Third, peptide epitopes may be fused to the protein to facilitate protein production and identification. For example, a C-terminal His6 tag may be included to facilitate purification and to provide an epitope for antibody-based detection within the device. The recombinant acetylcholine receptor protein may have a predicted isoelectric point near 5.3.

### Nicotine Conjugate as Immobilized Agent

In one aspect, the assay device may further comprise a nicotine conjugate immobilized in the test visualization zone as an immobilized agent. The nicotine conjugate may consist of nicotine or a structurally related ligand covalently bound to a carrier protein such as bovine serum albumin (BSA) or other suitable proteins. The immobilized agent may be capable of selectively binding the recombinant acetylcholine receptor protein in the absence of free ligand. In a buffered solution, the recombinant acetylcholine receptor protein and the nicotine conjugate may form a stable receptor-ligand complex, and the displacement of this complex by competing ligands may serve as the basis of analyte detection.

The nicotine conjugate may comprise nicotine-N-β-D-glucuronide (NG) coupled to a carrier protein such as modified bovine serum albumin. The NG-protein conjugates may be prepared by first attaching a flexible diamino spacer, such as 1,4-diaminobutane (DAB), to an inert carrier protein, such as BSA. The DAB spacer may be coupled directly to BSA using a crosslinking agent, such as N-(3-dimethylaminopropyl)-N′-ethylcarbodiimide hydrochloride (EDAC). To improve the efficiency of the spacer addition reaction, it may be useful to first acetylate the BSA to minimize undesired crosslinking and also to perform the reaction at a pH value in the range 6.0 to 7.5 to optimize carrier protein solubility. The NG ligand group may then be attached to the spacer-modified carrier protein using EDAC. The 1,4-diaminobutane linker may project the NG ligand away from the surface of the carrier protein, allowing it to penetrate into the hydrophobic binding site of the receptor. The NG moiety may also be attached to the protein using a longer flexible Jeffamine ED-600 linker to produce a fully functional nicotine conjugate capable of binding the recombinant acetylcholine receptor protein.

As may be appreciated by a person skilled in the art, any aliphatic or polyethylene-based spacer containing between 4 and 20 carbon atoms may be used to make functional spacer groups on the carrier protein. Longer polyethylene-based linkers may also be used to produce active nicotine conjugates. Other nicotine derivatives modified at the 1-position of the pyridine ring may also be useful to make binding conjugates suitable for use as the immobilized agent.

The invention may further include insights relating to the design of functional nicotine conjugates. Attachment of protein through the amine group on 3′-aminomethyl nicotine may yield an inactive conjugate that does not bind the recombinant acetylcholine receptor protein. This may be consistent with structural studies showing that the pyrrolidine ring of nicotine occupies a buried hydrophobic pocket within the receptor, whereas the pyridine ring is more solvent-exposed. Functional conjugates may therefore require attachment through the pyridine ring rather than the pyrrolidine moiety. Coupling the protein through the glucuronide carboxyl group of nicotine-N-β-D-glucuronide may produce an active nicotine conjugate that binds strongly to the recombinant acetylcholine receptor protein. To those skilled in the art, it may be apparent that attachment through the pyridine-associated functionality is necessary to preserve binding activity and enable effective assay performance.

### Receptor-Reporter Conjugate

A reporter group, such as gold nanoparticles or an enzyme (e.g., horseradish peroxidase), may be conjugated to the recombinant acetylcholine receptor protein to form a receptor-reporter conjugate. The conjugation may occur by passive adsorption, covalent coupling, or other suitable means. The recombinant acetylcholine receptor protein, which may have a predicted isoelectric point near 5.3, may be attached to the gold nanoparticles using passive adsorption in a dilute potassium citrate buffer. The remaining nonspecific protein binding sites on the nanoparticles may then be blocked with BSA protein. The recombinant acetylcholine receptor protein may be attached to colloidal gold or other reporter nanoparticles such as quantum dots and fluorescent polystyrene nanospheres. Alternatively, the recombinant acetylcholine receptor protein may be attached to the nanoparticles by direct chemical attachment.

### Operation of the Lateral Flow Assay Device

During operation, the mobile agent encounters the immobilized agent as the liquid sample flows along the porous strip. In the absence of a nicotinic acetylcholine receptor ligand in the sample, the receptor-reporter conjugate binds to the immobilized agent, producing a visually detectable signal at the test visualization zone. The amount of reporter captured at the immobilized agent may correspond to the extent of receptor binding.

If the sample contains a nicotinic acetylcholine receptor ligand, such as nicotine, acetylcholine, neonicotinoid pesticide, anatoxin, dihydroanatoxin, cytisine, cyclic imines, or other structurally related compound, the ligand may compete with the immobilized agent for binding to the receptor-reporter conjugate. When a sample containing an analyte capable of binding the recombinant acetylcholine receptor protein is mixed with the receptor-reporter conjugate, the analyte may compete with the immobilized nicotine conjugate for receptor binding, resulting in a decrease in bound reporter that may be proportional to the analyte concentration. This competition may inhibit the binding of the receptor-reporter conjugate to the immobilized agent, leading to a reduction in the visually detectable signal. Therefore, the intensity of the signal may be inversely proportional to the concentration of the nicotinic acetylcholine receptor ligand in the sample. The resulting weak or absent signal may indicate the presence of nicotine-like compounds in the sample.

### Running Buffer Formulation

The liquid sample may comprise an aqueous running buffer solution optimized for promoting selective binding and rapid capillary-driven flow through the nitrocellulose membrane. In various embodiments, the running buffer suitable for promoting selective binding may include a tris(hydroxymethyl)aminomethane (Tris) buffer, sodium chloride, a non-ionic surfactant such as Tween 20, and a blocking protein such as bovine serum albumin. The running buffer may be optimized to maximize the specific binding of the receptor to the immobilized conjugate while minimizing non-specific binding and background signal.

In particular embodiments, the running buffer solution may comprise 40 millimolar (mM) of tris(hydroxymethyl)aminomethane buffer solution, 300 mM of sodium chloride, 0.1 percent of Tween 20 detergent, and 0.1 percent of bovine serum albumin. Each component of the running buffer may serve a distinct functional purpose in promoting assay performance. The pH is about 7.5. In addition, 0.1% sodium Azide may be added, which may serve as a preservation agent.

The 40 mM Tris component may serve as the primary pH buffering system. Tris may exhibit a pKa of approximately 8.07 at 25 degrees Celsius, providing buffering capacity in the physiological range of approximately pH 7 to 9.2. This concentration may maintain the solution at a slightly alkaline pH, typically around pH 7.6 to 8.0, which may be optimal for receptor-ligand interactions on nitrocellulose membranes.

The 300 mM sodium chloride component may provide critical functions in the running buffer. Physiologically, this concentration may create an isotonic environment that prevents osmotic damage to proteins and membrane artifacts. More importantly, salt at this concentration may provide ionic strength to the buffer, which may help stabilize receptor-protein interactions and reduce non-specific electrostatic binding to the nitrocellulose membrane surface. The 300 mM concentration may fall within the typical range used in lateral flow buffers and may maintain osmolarity while supporting efficient capillary flow through the porous membrane structure. The specific ionic strength may influence both the speed and quality of analyte migration along the strip.

The 0.1 percent Tween 20 (polysorbate 20) component may represent a standard concentration for lateral flow applications and may serve multiple critical functions in the assay. First, Tween 20 may act as a blocking agent by reducing non-specific adsorption of assay reagents, particularly the receptor-reporter conjugates, to the nitrocellulose membrane and other solid surfaces within the strip. Second, Tween 20 may improve wetting characteristics of the buffer, ensuring even distribution of the sample across the membrane and promoting uniform capillary flow. Third, Tween 20 may act as an emulsifier, stabilizing any lipophilic components that may be present in the assay system. At 0.1 percent, the concentration may be sufficient to provide blocking efficacy while remaining gentle enough to avoid disrupting the specific receptor-ligand interactions that form the basis of the assay.

The 0.1 percent bovine serum albumin (BSA) component may serve as an additional passive blocking agent with distinct properties from Tween 20. BSA may be a large protein that physically coats exposed surface areas on the nitrocellulose membrane, preventing unbound proteins from non-specifically adhering to these surfaces. BSA may function by providing alternate binding sites that preferentially capture interfering molecules rather than allowing them to interfere with specific assay reactions. At 0.1 percent, BSA may work synergistically with Tween 20 to create a comprehensive blocking environment. Unlike Tween 20, which may primarily address hydrophobic interactions, BSA may also reduce electrostatic and other protein-protein non-specific interactions, making the combination of both blocking agents particularly effective. Additionally, BSA may function as a stabilizer, helping to prevent denaturation of the receptor and other proteins in the buffer during storage and use.

In embodiments, an alternative formulation for the running buffer may include 50 mM Tris, 250 mM sodium chloride, 0.05% Tween and 2 mM Zinc chloride, and 0.1% Sodium Azide with a pH of 7.5. Other formulations are also contemplated. In one example, a 1× running buffer at pH 7.5 may include:
i. Tris (tris(hydroxymethyl)aminomethane): from about 10 mM to about 100 mM
ii. NaCl (Sodium Chloride): from about 10 nM to about 400 mM
iii. Tween 20: from about 0.01% to about 1%
iv. Bovine Serum Albumin: from about 0.1% to about 1%
v. Optionally, Sodium Azide may be added to reach a final concentration between about 0.05% and 1%.

In embodiments, the formulation of the running buffer solution may be diluted or otherwise modified to reach a range of concentrations from about 0.1× to about 10×.

The running buffer formulation may represent a system specifically optimized for lateral flow assays on nitrocellulose membranes. The specific concentrations may reflect optimization for rapid capillary-driven flow while maintaining high specificity and sensitivity. The combination of Tris, sodium chloride, Tween 20, and BSA may create a buffer system that maintains physiological pH optimal for receptor-ligand reactions, provides adequate ionic strength to promote proper protein-protein binding while minimizing electrostatic non-specific interactions, minimizes background signal through comprehensive blocking of non-specific binding sites, promotes even distribution and flow of sample through the membrane, stabilizes assay reagents throughout the assay duration, and supports the typical architecture of lateral flow test strips including sample pad, conjugate release pad, detection zones on nitrocellulose membrane, and absorbent pad. This buffer composition may be particularly well-suited for gold nanoparticle-based lateral flow assays, where controlling both non-specific binding and optimal capillary flow may be critical for achieving low limits of detection and high signal-to-noise ratios. The formulation may be used as the primary buffer component applied to the conjugate pad or used as the running buffer applied during the assay, depending on specific protocol requirements.

The receptor-reporter conjugate may also be preloaded onto a conjugate pad (the second zone) along the porous strip and may be rehydrated upon introduction of the liquid sample containing the running buffer. This configuration may allow for convenient storage and transport of the device while ensuring that the receptor remains active and available for binding upon rehydration.

### Configuration of the Lateral Flow Assay Device

In one embodiment, the assay may be configured as a competitive lateral flow device. The nicotine conjugate may be immobilized on a solid support such as a nitrocellulose membrane to create a test line within the test visualization zone. The porous strip may be laminated with a wicking pad on one end (the fourth zone) to facilitate the flow of liquid through the strip and a sample pad (the first zone) and conjugate pad (the second zone) to apply the sample to the porous strip. After lamination, the membranes may be cut into strips and stored in desiccated canisters.

During use, the receptor-reporter conjugates may be combined with the sample and running buffer and then applied to the first zone (sample pad). The liquid may then enter the device and flow through the strip by capillary action. As the mixture migrates along the porous strip, the receptor-reporter conjugate encounters the immobilized nicotine conjugate at the test line. In another embodiment, the receptor-reporter conjugate may be applied to the second zone (conjugate pad) after lamination and dried before the membranes are cut into strips. In this embodiment there may be no requirement to premix the nanoparticles with the sample; the sample may be applied directly to the strips.

The gold nanoparticle conjugates may be introduced directly into the liquid sample or applied in dried form onto a conjugate pad where they may rehydrate upon sample flow. The lateral flow assay device may be housed in a cassette for convenient handling and use.

### Control Zone

The device may further include a control zone comprising a separate immobilized agent, which may serve to confirm proper operation of the test regardless of analyte concentration. The porous strip may be sprayed with a protein that can bind the receptor-reporter conjugate, such as anti-His6 antibody, or another protein conjugate that can bind a second independent nanoparticle type.

In one approach, a second population of labeled nanoparticles may be incorporated into the device, formulated to bind a conjugate immobilized at the control line and generate a consistent signal independent of analyte concentration. As an example, the control line may be sprayed with biotinylated BSA and the second nanoparticle type could be streptavidin-coated gold nanoparticles, which may capture the second population of labeled nanoparticles (e.g., streptavidin-coated nanoparticles).

In an alternative approach, antibodies that recognize the recombinant acetylcholine receptor protein may be immobilized at the control line. For example, anti-His6 antibodies may be used to bind a C-terminal His6 tag on the recombinant acetylcholine receptor protein. In this embodiment, only a single population of labeled nanoparticles may be required.

### Alternative Embodiments: Microwell Format

Alternative embodiments may employ a microwell plate format wherein the immobilized agent may be coated on the surface of the wells, and the recombinant acetylcholine receptor protein may be introduced to each well. Signal generation and analysis may proceed via optical absorbance or fluorescence measurements.

In one embodiment, the assay may be a 96-well microplate-based assay. The NG-BSA conjugate may be coated onto the surface of the ELISA plate wells. The recombinant acetylcholine receptor protein may be mixed with liquid samples and buffer in the coated wells. The buffer used may maximize the binding of the receptor to the conjugate during the assay procedure. Using this buffer for all subsequent incubation and washing steps of the procedure may enhance the specific detection signal in the assay. If no analytes are present in the sample, then the receptor may specifically bind to the nicotine conjugate. If analytes are present in the sample, they may prevent the receptor from binding to the nicotine conjugate. Therefore, the amount of receptor retained in the wells after washing may be approximately inversely proportional to the amount of analyte in the sample.

The amount of receptor bound in the well may be determined by adding dilute anti-His6 antibody (produced in rabbits) to the wells. After incubating for 30 minutes, the wells may be washed with buffer, and then dilute goat anti-rabbit-horseradish peroxidase conjugate may be added to the wells and incubated for 30 minutes before washing the wells again and adding 3,3′,5,5′-Tetramethylbenzidine (TMB) reagent to detect the bound peroxidase enzyme.

In another embodiment, the surface of the wells may be coated with recombinant acetylcholine receptor protein (either directly or indirectly by using a well coated with an antibody that binds the receptor). The nicotine conjugate may be mixed with liquid samples and buffer in the coated wells. The amount of nicotine conjugate bound in the well may be determined by adding an antibody that binds to the conjugate protein to the wells.

### Applications and Sample Types

In both formats, the assay may be suitable for the detection of a broad range of analytes, including but not limited to, nicotine, neonicotinoids, anatoxin, dihydroanatoxin, cytisine, cyclic imines, and other ligands capable of binding the recombinant acetylcholine receptor protein. The assay may be suitable for detecting these target ligands in sample types such as recreational water, groundwater, seawater, wastewater, food extracts, and environmental swabs.

### Kits and Advantages

The invention may provide for kits including a lateral flow assay device, one or more containers of recombinant acetylcholine receptor protein (liquid or lyophilized), buffer, and instructions for use. The device may be configured to provide rapid and sensitive detection suitable for laboratory, field, or point-of-care testing, and may avoid the use of radioactive materials, live cells, or complex analytical instrumentation. The invention may enable detection of receptor-binding compounds in liquid samples through modulation of a detectable signal, providing a versatile platform suitable for both lateral flow and microwell formats, and overcoming many of the disadvantages associated with existing methods for detecting nicotinic acetylcholine receptor ligands.

### EXAMPLES

The following examples illustrate specific embodiments of the invention and demonstrate the preparation and use of the lateral flow assay device and microwell assay format for detecting nicotinic acetylcholine receptor ligands. These examples are provided for illustrative purposes and should not be construed as limiting the scope of the invention.

### Example 1: Production of Recombinant Acetylcholine Receptor Protein

This example may describe the production of a recombinant acetylcholine receptor protein suitable for use as the mobile agent in the lateral flow assay device.

A DNA sequence encoding the AcAChBP protein (SEQ ID NO: 1) may be inserted into the pFastBac1 plasmid at the EcoRI/HindIII cloning sites. DNA encoding a signal sequence and a hexahistidine (His6) sequence may be fused to the amino and carboxy termini of the protein, respectively, during the subcloning process. The signal sequence may promote efficient secretion and proper disulfide bond formation, while the His6 tag may facilitate purification and antibody-based detection within the device.

The expression construct may be transfected into High Five insect cells. The recombinant acetylcholine receptor protein may be obtained from the supernatant of transfected insect cell culture. The receptor may be purified from the culture supernatant using column chromatography, including an immobilized Ni column followed by size exclusion chromatography (Chromdex 200), into 50 millimolar (mM) Tris-HCl, 500 mM NaCl, 5 percent glycerol, pH 8.0.

The purified solution, containing 0.2 milligrams per milliliter (mg/mL) protein, may be aliquoted into 0.2 milliliter (mL) portions and frozen at negative 80 degrees Celsius for storage.

The amino acid sequence of the AcAChBP protein (SEQ ID NO: 1) may be as follows:

MLLVNQSHQGFNKEHTSKMVSAIVLYVLLAAAAHSAFAQANLMRLKSDL
FNRSPMYPGPTKDDPLTVTLGFTLQDIVKVDSSTNEVDLVYYEQQRWKL
NSLMWDPNEYGNITDFRTSAADIWTPDITAYSSTRPVQVLSPQIAVVTH
DGSVMFIPAQRLSFMCDPTGVDSEEGVTCAVKFGSWVYSGFEIDLKTDT
DQVDLSSYYASSKYEILSATQTRQVQHYSCCPEPYIDVNLVVKFRERRA
GNGFFRNLFDAAHHHHHH

The N-terminal signal sequence (MLLVNQSHQGFNKEHTSKMVSAIVLYVLLAAAAHSAFA), which may be cleaved off the mature protein fragment during processing, is shown at the beginning of the sequence. The hexahistidine (His6) tag on the C-terminus of the protein (HHHHHH), which may facilitate purification and antibody detection, is shown at the end of the sequence.

### Example 2: Preparation of Receptor-Reporter Conjugate Using Gold Nanoparticles

This example may describe the preparation of a receptor-reporter conjugate comprising the recombinant acetylcholine receptor protein conjugated to gold nanoparticles for use as the mobile agent.

One hundred microliters (μL) of 22 mM potassium citrate, pH 5.0, containing 0.1 percent sodium azide may be added to 1 mL of colloidal gold at an optical density of 1 (40 nanometer particles). Twenty microliters of a 0.2 mg/mL recombinant acetylcholine receptor protein stock solution may then be added, and the mixture may be thoroughly mixed. The suspension may be incubated for 30 minutes at room temperature to allow passive adsorption of the protein to the gold nanoparticles.

After incubation, 200 μL of 22 mM potassium citrate, pH 5.0, containing 5 percent bovine serum albumin (BSA) and 0.1 percent sodium azide may be added and thoroughly mixed. The BSA may block remaining nonspecific protein binding sites on the nanoparticles. The mixture may be centrifuged at 1,400 times gravity (g) for 30 minutes. Following centrifugation, the supernatant may be removed, and the pellet may be resuspended in 100 μL of 22 mM potassium citrate, pH 5.0, containing 5 percent BSA and 0.1 percent sodium azide to produce the 10 optical density (OD) nanoparticle suspension containing the receptor-reporter conjugate.

### Example 3: Preparation of Nicotine Conjugate as Immobilized Agent

This example may describe the preparation of a nicotine conjugate suitable for use as the immobilized agent in the test visualization zone.

A total of 1.25 mL of 40 mg/mL acetylated bovine serum albumin (ac-BSA), previously dialyzed into ultrapure water, may be diluted with 3.75 mL of 1 molar (M) HEPES buffer at pH 6.5. EDAC (130 milligrams, N-(3-dimethylaminopropyl)-N′-ethylcarbodiimide hydrochloride) may be added, gently mixed until dissolved, and the mixture may be incubated at room temperature for 15 minutes. Then, 50 μL of 1,4-diaminobutane (DAB) may be added slowly, mixed, and the reaction may be allowed to proceed for 3 hours at room temperature. The resulting DAB-ac-BSA may be dialyzed seven times against 4 liters of 0.05 M MES buffer, pH 6.5, at 4 degrees Celsius to remove excess reagents. Protein concentration may be determined by absorbance at 280 nanometers (nm) using a molar extinction coefficient of 43,800 per molar per centimeter (M⁻¹ cm⁻¹).

A separate reaction mixture may be prepared by combining 300 μL of 1 M HEPES, pH 6.5; 150 μL of 0.5 M MES, pH 5.2; 1,200 μL of 8 mg/mL DAB-ac-BSA; 150 μL of 4 mg/mL nicotine-N-β-D-glucuronide; 150 μL of 0.16 M EDAC; and 75 μL of 5 M sodium chloride (NaCl). This mixture may be incubated for 3 hours at room temperature to allow coupling of the nicotine-N-β-D-glucuronide to the DAB-ac-BSA carrier protein. The reaction product may then be dialyzed in 1× phosphate-buffered saline (PBS) at 4 degrees Celsius with stirring for 6 days, with the dialysis buffer replaced every 8 to 24 hours to remove impurities. After dialysis, the protein concentration may be measured, and sodium azide may be added to a final concentration of 0.05 percent to produce the nicotine conjugate.

### Example 4: Detection of Nicotine Using Spotted Lateral Flow Strips

This example may demonstrate the specific binding of the receptor-reporter conjugate to an immobilized nicotine conjugate on lateral flow strips and the competitive inhibition of binding by nicotine in the sample.

A 1 μL aliquot of dialyzed nicotine-N-β-D-glucuronide protein conjugate (3.8 mg/mL) may be spotted onto blank lateral flow strips comprising nitrocellulose membrane and allowed to dry for 15 minutes to create a test visualization zone. A suspension consisting of 5 μL of receptor-coated gold nanoparticles (10 OD at 530 nm, prepared as in Example 2) and 145 μL of 1× Running Buffer (50 mM Tris-Cl, 250 mM NaCl, 0.2 percent BSA, 0.05 percent Tween-20, pH 7.5) may be prepared in a micro flat-bottom plastic tube. Each lateral flow strip may be immersed in the mixture and permitted to flow up the strip by capillary action for 15 minutes.

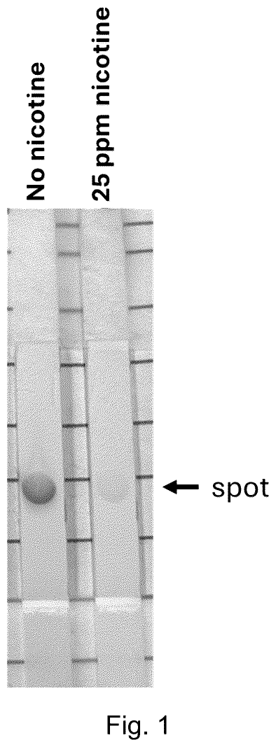

In the absence of nicotine (0 parts per million, ppm), the receptor-reporter conjugates may visibly bind to the spotted nicotine conjugate, producing a visually detectable signal at the test visualization zone (left strip in FIG. 1). When nicotine (25 ppm) may be added to the running buffer, binding of the coated gold to the spotted conjugate may be substantially inhibited or abolished, resulting in reduced signal intensity at the test visualization zone (right strip in FIG. 1). This example may demonstrate that the presence of a nicotinic acetylcholine receptor ligand in the sample competes with the immobilized agent for binding to the receptor-reporter conjugate, thereby reducing the visually detectable signal.

### Example 5: Fabrication of a Complete Lateral Flow Assay Device with Control Zone

This example may describe the fabrication of a complete lateral flow assay device comprising both a test visualization zone and a control zone, housed in a plastic cassette.

A solution of nicotine-N-β-D-glucuronide-ac-BSA at 2 mg/mL in phosphate-buffered saline (pH 7.4) containing 5 percent sucrose and 0.1 percent sodium azide may be sprayed onto 30-centimeter (cm) plastic-backed nitrocellulose cards at 1 μL/cm to form the test line in the test visualization zone. A parallel line of biotinylated BSA (0.5 mg/mL) in the same buffer, also applied at 1 μL/cm, may then be sprayed to form the control line in the control zone.

The cards may be laminated with a gold conjugate pad (the second zone) and a wick (the fourth zone). A mixture of 0.25 mL of streptavidin-coated gold nanoparticles (10 OD) and 1 mL of receptor-coated gold nanoparticles (prepared as in Example 2) may be prepared and sprayed onto the gold conjugate pad at 6 μL/cm. The cards may be dried for 60 minutes at 37 degrees Celsius.

The dried, laminated cards may be cut into 65 millimeters by 4.5 millimeters (mm) strips, assembled, and packaged into plastic lateral flow cassettes. The completed strips may be stored at room temperature in closed boxes containing desiccant. Alternatively, each strip may be placed into a plastic cassette housing containing a sample port and a viewing window before storage. This configuration may allow for convenient handling and use of the lateral flow assay device.

### Example 6: Detection of Anatoxin-a in Water Using a Complete Lateral Flow Device

This example may demonstrate the use of the lateral flow assay device housed in a plastic cassette for detecting anatoxin-a, a cyanobacterial neurotoxin, in water samples.

A 75 μL aliquot of 2× Running Buffer (100 mM Tris-Cl, 500 mM NaCl, 0.4 percent BSA, 0.1 percent Tween-20, 0.1 percent sodium azide, pH 7.5) may be combined with either 75 μL of water containing 100 parts per billion (ppb) anatoxin-a or 75 μL of water containing no anatoxin-a in a micro flat-bottom tube. The resulting mixture may be transferred to the sample port of a plastic cassette housing a lateral flow device (prepared as in Example 5). After a 15-minute incubation during which the sample may migrate along the strip by capillary action, the intensities of the control and test line signals may be measured.

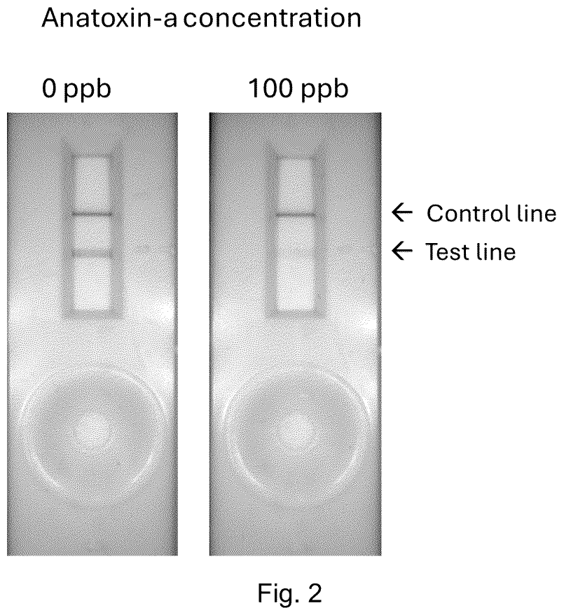

The presence of anatoxin-a in the sample may result in a marked decrease in test line intensity (lower arrow in FIG. 2) relative to the signal produced using a water sample containing no toxin (left device in FIG. 2). The control line (upper arrow in FIG. 2) may remain unaffected by the presence of anatoxin-a, confirming proper operation of the device. This example may demonstrate that the lateral flow assay device may be suitable for detecting nicotinic acetylcholine receptor ligands such as anatoxin-a in environmental water samples.

### Example 7: Detection of Dihydroanatoxin-a in Water Using Lateral Flow Strips

This example may demonstrate the detection of dihydroanatoxin-a, another cyanobacterial neurotoxin, in water samples using lateral flow strips.

A 100 μL aliquot of 2× Running Buffer (100 mM Tris-Cl, 500 mM NaCl, 0.4 percent BSA, 0.1 percent Tween-20, 0.1 percent sodium azide, pH 7.5) may be combined with either 100 μL of water containing 32 ppb dihydroanatoxin-a or 100 μL of water containing no dihydroanatoxin-a in a micro flat-bottom plastic tube. An additional 4 μL of receptor-coated gold nanoparticles and 1 μL of streptavidin-coated gold nanoparticles (10 OD at 530 nm) may then be added, and the mixture may be combined thoroughly. A lateral flow strip (containing no dried gold on the conjugate pad or plastic cassette housing) may then be immersed in the mixture. After a 15-minute incubation during which the sample may flow up the strip, the intensities of the control and test line signals may be measured.

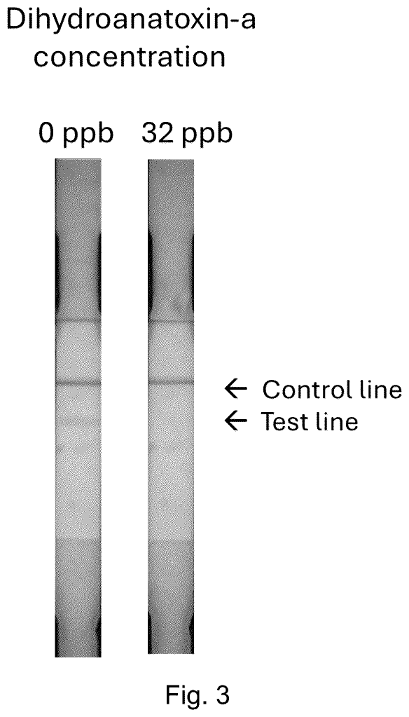

The presence of dihydroanatoxin-a in the sample may result in a marked decrease in test line signal intensity (right strip in FIG. 3) compared to the test performed using a water sample containing no dihydroanatoxin-a (left strip in FIG. 3). This example may demonstrate that the assay may be configured to detect dihydroanatoxin-a by premixing the receptor-reporter conjugates with the sample before application to the lateral flow strip.

### Example 8: Detection of Nicotine in Water Using Lateral Flow Strips

This example may demonstrate the detection of nicotine in water samples using lateral flow strips.

A 100 μL aliquot of 2× Running Buffer (100 mM Tris-Cl, 500 mM NaCl, 0.4 percent BSA, 0.1 percent Tween-20, 0.1 percent sodium azide, pH 7.5) may be combined with either 100 μL of water containing 17 ppm nicotine or 100 μL of water containing no nicotine in a micro flat-bottom tube. An additional 4 μL of receptor-coated gold nanoparticles and 1 μL of streptavidin-coated gold nanoparticles (10 OD at 530 nm) may be added, and the mixture may be combined thoroughly. A lateral flow strip (containing no dried gold on the conjugate pad) may then be immersed in the mixture. After a 15-minute incubation during which the sample may flow up the strip, the intensities of the control and test line signals may be measured.

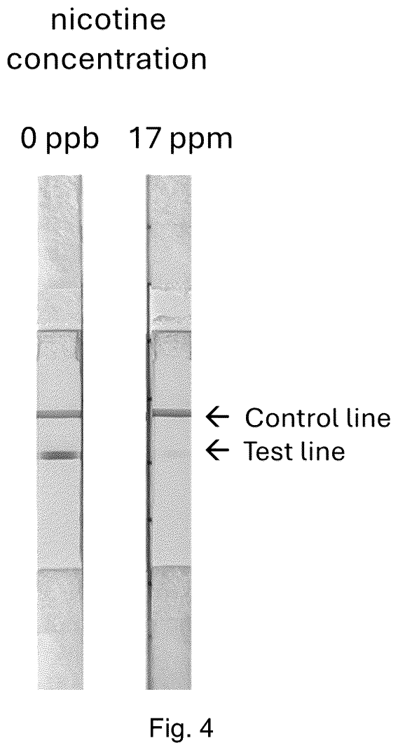

The presence of nicotine in the sample may result in a marked decrease in test line signal intensity (right strip in FIG. 4) relative to the signal produced using a water sample containing no nicotine (left strip in FIG. 4). This example may demonstrate that the lateral flow assay device may be suitable for detecting nicotine at concentrations relevant to environmental and recreational water testing.

### Example 9: Detection of Imidacloprid Pesticide in Water Using Lateral Flow Strips

This example may demonstrate the detection of imidacloprid, a neonicotinoid pesticide that binds to nicotinic acetylcholine receptors, in water samples using lateral flow strips.

A 100 μL aliquot of 2× Running Buffer (100 mM Tris-Cl, 500 mM NaCl, 0.4 percent BSA, 0.1 percent Tween-20, 0.1 percent sodium azide, pH 7.5) may be combined with either 100 μL of water containing 1250 ppb imidacloprid or 100 μL of water containing no imidacloprid in micro flat-bottom tubes. An additional 4 μL of receptor-coated gold nanoparticles and 1 μL of streptavidin-coated gold nanoparticles may be added, and the mixture may be combined thoroughly. A lateral flow strip (containing no dried gold on the conjugate pad) may then be immersed in the mixture. After a 15-minute incubation during which the sample may flow up the strip, the intensities of the control and test line signals may be measured.

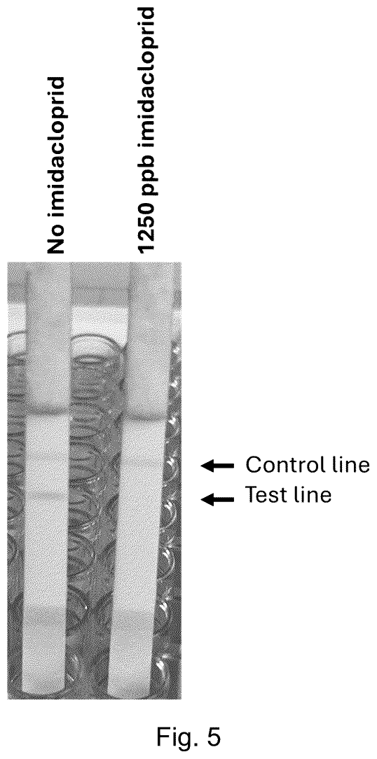

The presence of imidacloprid may substantially abolish the test line intensity (right strip in FIG. 5) relative to the signal produced using a water sample containing no imidacloprid (left strip in FIG. 5). This example may demonstrate that the lateral flow assay device may be suitable for detecting neonicotinoid pesticides such as imidacloprid in environmental samples, which may be useful for monitoring pesticide contamination in water sources.

### Example 10: Detection of Nicotine Using Microwell Plate Format

This example may demonstrate an alternative embodiment employing a microwell plate format for detecting nicotine in liquid samples.

96-well plate wells may be coated with 100 μL of 5 micrograms per milliliter (μg/mL) nicotine N-β-D-glucuronide ac-BSA in 0.1 M sodium carbonate pH 9.0 for 16 hours and subsequently blocked with 1 percent BSA. The recombinant acetylcholine receptor protein (AcAChBP) stock solution (0.2 mg/mL) may be diluted 1:100 into 50 mM Tris-Cl, 250 mM NaCl, 0.2 percent BSA, and 0.05 percent Tween-20 at pH 7.5, with the buffer either lacking nicotine or supplemented with 0.5 μg/μL nicotine. A volume of 100 μL of the diluted protein may be added to each well, and the plates may be incubated for 30 minutes at room temperature. The wells may then be washed three times with the same buffer.

Anti-His6 antibody may be diluted 1:30,000 in the same buffer, after which 100 μL may be added to each well. Following a 30-minute incubation at room temperature, the wells may be washed three times. Goat anti-rabbit horseradish peroxidase (HRP) conjugate may be diluted 1:100 in the same buffer, and 100 μL may be dispensed into each well. After a 30-minute incubation at room temperature, the wells may be washed three times with the same buffer.

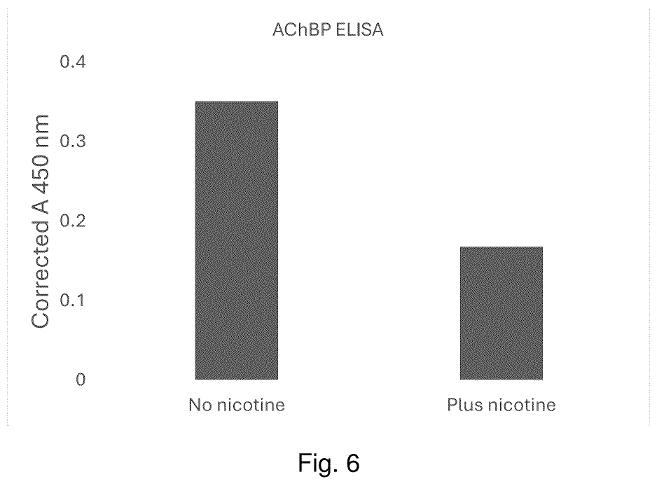

TMB substrate (3,3′,5,5′-Tetramethylbenzidine, 100 μL per well) may then be added, and color development may proceed for 20 minutes at room temperature. The reaction may be stopped by adding 100 μL of stop solution to each well, and absorbance may be measured at 450 nm (FIG. 6).

In this microwell assay format, mixing the recombinant acetylcholine receptor protein with water containing 0.5 μg/μL nicotine (right bar in FIG. 6) may cause a lower enzymatic signal than the signal caused by mixing the receptor protein with water containing no nicotine (left bar in FIG. 6). The amount of receptor bound to the immobilized nicotine conjugate may be proportional to the observed absorbance at 450 nm. This example may demonstrate that the invention may also be configured as a microwell plate-based assay suitable for laboratory screening applications, providing signal generation and analysis via optical absorbance measurements.

These examples may collectively demonstrate the versatility of the invention for detecting a broad range of nicotinic acetylcholine receptor ligands, including nicotine, anatoxin-a, dihydroanatoxin-a, and neonicotinoid pesticides, in various sample types such as water and environmental samples. The examples may further demonstrate that the invention may be configured in both lateral flow and microwell formats, offering flexibility for field testing and laboratory applications.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A lateral flow assay device for detecting a presence of a nicotinic acetylcholine receptor ligand in a liquid sample, the lateral flow assay device comprising:
a porous strip with a plurality of zones located along thereof as follows:
a first zone for depositing the liquid sample,
a third zone comprising, in turn, a test visualization zone comprising a nicotine conjugate as an immobilized agent, and a control zone, and
a fourth zone configured to absorb the liquid sample therein, and
a detectable recombinant acetylcholine receptor protein as a mobile agent.

2. The lateral flow assay device, as in claim 1, wherein the recombinant acetylcholine receptor protein is present on a second zone of the porous strip between the first zone and the third zone thereof.

3. The lateral flow assay device, as in claim 1, wherein the recombinant acetylcholine receptor protein is configured to be present in or added to the liquid sample prior to depositing the liquid sample onto the first zone of the porous strip.

4. The lateral flow assay device, as in claim 3, wherein the recombinant acetylcholine receptor protein is provided in a liquid form or in a lyophilized powder form, the recombinant acetylcholine receptor protein is stored in a container configured for mixing the recombinant acetylcholine receptor protein with the liquid sample, wherein the container with the recombinant acetylcholine receptor protein and the porous strip form a test kit for detecting the nicotinic acetylcholine receptor ligand.

5. The lateral flow assay device, as in claim 1, wherein the first zone and the fourth zone are located on opposite sides of the porous strip.

6. The lateral flow assay device, as in claim 1, wherein the nicotinic acetylcholine receptor ligand is acetylcholine.

7. The lateral flow assay device, as in claim 1, wherein the nicotinic acetylcholine receptor ligand is a neonicotinoid pesticide.

8. The lateral flow assay device, as in claim 1, wherein the nicotinic acetylcholine receptor ligand is anatoxin, anatoxin-a, Dihydroanatoxin-a, nicotine, bungarotoxin, or a cyclic imine.

9. The lateral flow assay device, as in claim 8, wherein the recombinant acetylcholine receptor protein is a purified recombinant Aplysia californica, the nicotinic acetylcholine receptor ligand is Dihydroanatoxin-a, and the nicotine conjugate is a conjugate of bovine serum albumin with nicotine or a derivative thereof.

10. The lateral flow assay device, as in claim 1, wherein the porous strip is a nitrocellulose membrane configured to promote capillary-driven flow of the liquid sample therethrough, the liquid sample comprises an aqueous running buffer solution, which in turn, comprises a tris(hydroxymethyl)aminomethane buffer solution, Sodium Chloride, Tween 20 detergent, and bovine serum albumin.

11. The lateral flow assay device, as in claim 10, wherein the running buffer solution comprises between 10 and 400 mM of tris(hydroxymethyl)aminomethane buffer solution, between 10 and 400 mM of Sodium Chloride, between 0.01 and 1 percent of Tween 20 detergent, and between 0.1 and 1 percent of bovine serum albumin.

12. The lateral flow assay device, as in claim 1, wherein the recombinant acetylcholine receptor protein is conjugated to a reporter group selected to generate a visually detectable signal at the test visualization zone of the porous strip.

13. The lateral flow assay device, as in claim 12, wherein the presence of the nicotinic acetylcholine receptor ligand causes a reduction in the visually detectable signal, thereby a concentration of the nicotinic acetylcholine receptor ligand is inversely proportional to a strength of the visually detectable signal.

14. A method of detecting a nicotinic acetylcholine receptor ligand, the method comprises the following steps:
a. providing a liquid sample suspected of containing the nicotinic acetylcholine receptor ligand,
b. adding a recombinant acetylcholine receptor protein to the liquid sample,
c. exposing the mixture of step (b) to an immobilized agent comprising a nicotine conjugate, thereby generating a visually detectable signal at the location of the immobilized agent,
d. detecting a presence of the nicotinic acetylcholine receptor ligand in the liquid sample by observing the visually detectable signal of step (c),
wherein the recombinant acetylcholine receptor protein is conjugated to a reporter group selected to generate the visually detectable signal, and
wherein the presence of the nicotinic acetylcholine receptor ligand causes a reduction in the visually detectable signal, thereby a concentration of the nicotinic acetylcholine receptor ligand is inversely proportional to an intensity of the visually detectable signal.

15. The method of detecting a nicotinic acetylcholine receptor ligand, as in claim 14, wherein at least steps (c) and (d) are performed as part of a high-throughput test, or an enzyme-linked immunosorbent assay (ELISA) test.

16. The method of detecting a nicotinic acetylcholine receptor ligand, as in claim 15, wherein the high-throughput test is conducted on a plate with a plurality of wells therein.

17. A test device for detecting a nicotinic acetylcholine receptor ligand in a liquid sample, the test device is configured to facilitate the detecting of claim 14, the test device comprises:
an application zone configured for receiving the liquid sample,
a support structure comprising a nicotine conjugate as an immobilized agent,
wherein the test device is further configured to apply a recombinant acetylcholine receptor protein as a mobile agent to the liquid sample, followed by a transfer thereof to the support structure, wherein the recombinant acetylcholine receptor protein is conjugated to a reporter group selected to generate a visually detectable signal, thereby forming a receptor-reporter conjugate,
wherein, in the absence of the nicotinic acetylcholine receptor ligand, the receptor-reporter conjugate binds to the immobilized agent, thereby generating the visually detectable signal on the support structure,
wherein, the presence of the nicotinic acetylcholine receptor ligand inhibits the binding of the receptor-reporter conjugate and reduces the intensity of the visually detectable signal,
thereby the intensity of the visually detectable signal inversely correlates to a concentration of the nicotinic acetylcholine receptor ligand in the liquid sample.


SEQUENCE LISTING (1 sequence)

SEQ ID NO: 1 - protein, 263 aa, Aplysia californica
MLLVNQSHQGFNKEHTSKMVSAIVLYVLLAAAAHSAFAQANLMRLKSDLFNRSPMYPGPTKDDPLTVTLGFTLQDIVKVDSSTNEVDLVYYEQQRWKLNSLMWDPNEYGNITDFRTSAADIWTPDITAYSSTRPVQVLSPQIAVVTHDGSVMFIPAQRLSFMCDPTGVDSEEGVTCAVKFGSWVYSGFEIDLKTDTDQVDLSSYYASSKYEILSATQTRQVQHYSCCPEPYIDVNLVVKFRERRAGNGFFRNLFDAAHHHHHH